(19) United States
(12) Patent Application Publication
(10) Pub. No.: US 2018/0117266 A1
(43) Pub. Date: May 3, 2018

(54) APPARATUS AND METHOD FOR NEUROLOGICAL TREATMENT

(71) Applicant: Monteris Medical Corporation, Plymouth, MN (US)

(72) Inventors: Mark GRANT, Winnipeg (CA); Keith SOOTSMAN, Kalamazoo, MI (US); Brooke REN, Maple Grove, MN (US); John SCHELLHORN, Edina, MN (US)

(21) Appl. No.: 15/573,103

(22) PCT Filed: Mar. 11, 2016
(86) PCT No.: PCT/US2016/022128

§ 371 Date: Nov. 9, 2017

Related U.S. Application Data
(60) Provisional application No. 62/132,970, filed on Mar. 13, 2015.
(60) Provisional application No. 62/209,109, filed on Aug. 24, 2015.

(51) Int. Cl. A61M 5/46; A61B 90/14; A61M 39/10; A61M 5/158; A61B 18/24

(52) U.S. Cl. CPC A61M 5/46; A61B 90/14; A61B 2018/00791; A61M 5/158; A61B 18/24; A61M 39/10

(57) ABSTRACT

An apparatus and method for performing neurological intervention may include a cylindrical body including a tapered threaded portion that screws into a pre-drilled hole in a subject. Additionally, the apparatus may include a connector having a probe lock-screw that is disposed on the cylindrical body, at an end opposite to the tapered threaded portion. The connector may further include an opening to receive a probe. The probe may be inserted into a subject via the apparatus, and a depth of insertion of the probe within the subject may be adjusted by the probe lock-screw.

### RELATED APPLICATIONS

This application is a U.S. National Stage Entry of International Application No. PCT/US2016/022128 filed Mar. 11, 2016 which claims the benefit and priority of U.S. Provisional Application No. 62/132,970, filed on Mar. 13, 2015, and U.S. Provisional Application No. 62/209,109, filed Aug. 24, 2015. All above-identified applications are hereby incorporated by reference in their entireties.

### BACKGROUND

The background description provided herein is for the purpose of generally presenting the context of the disclosure. Work of the presently named inventors, to the extent the work is described in this background section, as well as aspects of the description that may not otherwise qualify as prior art at the time of filing, are neither expressly nor impliedly admitted as prior art against the present disclosure.

Various forms of therapy can be applied within the body of a human or other mammalian subject, and in particular to the mammalian nervous system. For instance, therapy can be introduced to the mammalian nervous system by the application of energy from outside of the subject's body. In hyperthermia, ultrasonic or radio frequency energy is applied from outside the subject's body in order to heat certain tissues. The applied energy can be focused on a small region (spot) within the body, so as to heat the tissues at such spots at a temperature that is sufficient to create a desired therapeutic effect. In such a manner, this technique selectively destroys unwanted tissue within the body. For example, tumors or other unwanted tissues can be destroyed, without destroying the adjacent normal tissues, by applying heat at a temperature of approximately 50° C. to 70° C. Such a process is commonly referred to as ‘thermal ablation’. Other hyperthermia treatments include selectively heating tissues so as to selectively activate a drug or promote some other physiologic change in a selected portion of the subject's body. Other therapies, such as ultrasonic lithotripsy, employ the applied energy to destroy foreign objects or deposits within the body.

Furthermore, treatment for a particular disease or disorder can be introduced to the mammalian nervous system by application of a therapeutic agent to the mammalian nervous system, e.g., the mammalian brain. Therapeutic agents can be used to treat various disorders including neurodegenerative diseases, cancer, epilepsy and the like. However, the types of therapeutic agents that are useful for such therapies have been limited by challenges related to the delivery of such therapeutics across the blood-brain barrier. Although the blood-brain barrier is often compromised in certain advanced stage brain diseases, it is preferable to treat such disorders prior to the stage at which this disruption occurs. The introduction of the therapeutic agent to the desired location in the mammalian central nervous system (CNS), at an earlier stage in the disease process, requires more invasive methods of delivery.

Accordingly, there is a requirement for an apparatus and methods that enable the introduction of therapeutic agents into the mammalian central nervous system, for performing therapeutic treatments, thermal ablations, and the like, in a minimally invasive manner.

### SUMMARY

The present disclosure provides for an apparatus and corresponding methods that provide a stable platform for the introduction of surgical, therapeutic or diagnostic intervention into the central nervous system (CNS), and in particular into the brain of a mammalian subject. The apparatus provides stereotactic guidance for the placement and fixation of instruments for use in neurological procedures, and in particular for neurological procedures that are performed in conjunction with preoperative or perioperative monitoring such as magnetic resonance imaging (MRI) or computed tomography (CT) imaging. Moreover, the apparatus of the present disclosure is made from a material that is compatible with MRI or CT imaging systems.

The foregoing paragraphs have been provided by way of general introduction, and are not intended to limit the scope of the following claims. The described embodiments together, with further advantages, will be best understood by reference to the following detailed description taken in conjunction with the accompanying drawings.

### BRIEF DESCRIPTION OF THE DRAWINGS

Various embodiments of this disclosure that are provided as examples will be described in detail with reference to the following figures, wherein like numerals reference like elements, and wherein:

FIG. 1 illustrates according to an embodiment, an exemplary skull mounted bolt;

FIG. 2 depicts an alternate view of the exemplary skull mounted bolt of FIG. 1;

FIG. 3 depicts a cross-sectional view of the exemplary skull mounted bolt of FIG. 1;

FIG. 4 depicts according to an embodiment, the skull mounted bolt of FIG. 1 and a surgical probe;

FIG. 5 depicts an exploded view of the exemplary skull mounted bolt of FIG. 1;

FIGS. 6A-6C illustrate exemplary skull mounted bolts;

FIG. 7A depicts an exemplary driver;

FIG. 7B depicts an exemplary embodiment of the skull mounted bolt of FIG. 6A and the driver of FIG. 7A;

FIG. 8 illustrates according to one embodiment, an exemplary skull mounted bolt;

FIG. 9 illustrates according to one embodiment, an exemplary skull mounted bolt;

FIG. 10 illustrates an exemplary bolt driver;

FIG. 11 illustrates according to an embodiment, multiple skull mounted bolts affixed to the skull of a subject; and

FIG. 12 illustrates a block diagram of a computing device according to one embodiment.

### DETAILED DESCRIPTION

Exemplary embodiments are illustrated in the referenced figures of the drawings. It is intended that the embodiments and figures disclosed herein are to be considered illustrative rather than restrictive. No limitation on the scope of the technology and of the claims that follow is to be imputed to the examples shown in the drawings and discussed herein.

The embodiments are mainly described in terms of particular processes and systems provided in particular implementations. However, the processes and systems will operate effectively in other implementations. Phrases such as “an embodiment”, “one embodiment” and “another embodiment” may refer to the same or different embodiments. The embodiments will be described with respect to methods and compositions having certain components. However, the methods and compositions may include more or less components than those shown, and variations in the arrangement and type of the components may be made without departing from the scope of the present disclosure.

The exemplary embodiments are described in the context of methods having certain steps. However, the methods and compositions operate effectively with additional steps and steps in different orders that are not inconsistent with the exemplary embodiments. Thus, the present disclosure is not intended to be limited to the embodiments shown, but is to be accorded the widest scope consistent with the principles and features described herein and as limited only by appended claims.

Furthermore, where a range of values is provided, it is to be understood that each intervening value between an upper and lower limit of the range—and any other stated or intervening value in that stated range is encompassed within the disclosure. Where the stated range includes upper and lower limits, ranges excluding either of those limits are also included. Unless expressly stated, the terms used herein are intended to have the plain and ordinary meaning as understood by those of ordinary skill in the art. The following definitions are intended to aid the reader in understanding the present disclosure, but are not intended to vary or otherwise limit the meaning of such terms unless specifically indicated.

According to one embodiment of the present disclosure, the apparatus is a one-piece device that includes: a) a low-profile bolt having a tapered tread on its distal end that enables seamless insertion of the bolt into a skull or spinal cord of a subject, and b) a set of wings that allow the apparatus to be manipulated by a physician/surgeon such that the apparatus can be introduced in close proximity of the target tissue. A proximal end of the bolt is sized to accept an instrument for neurological intervention that can access the target tissue through the distal end of the bolt. Thus, the proximal end of the bolt is dimensioned in a manner such that multiple bolts can be placed in close proximity of one another. The distal end of the bolt is designed in a manner such that an internal opening (i.e., an internal diameter) at the distal end of the bolt is configured to receive instruments having dimensions of 3.3 mm or less. Additionally, the outside diameter of the distal end of the bolt is not greater than 6 mm in order to prevent injuries to the skull, and minimize the diameter of the drilled hole in the skull of the subject.

According to one embodiment, the apparatus of the present disclosure is a one-piece device that includes a low-profile bolt having a tapered thread on its distal end that enables seamless insertion of the bolt into a skull or spinal cord of a subject. Additionally, the device includes a built-in connector that is disposed on the bolt, wherein the connector provisions for connecting/inserting surgical instruments into the bolt. The connector may be a simple compression-type fitting, or a simple structure such as a collar.

According to one embodiment, the apparatus of the present disclosure is a two-piece device that includes: a) a low-profile bolt having a tapered tread on its distal end that enables seamless insertion of the bolt into a skull or spinal cord of a subject, b) a set of wings that allow the apparatus to be introduced in proximity of a target tissue, and c) a bushing that can be inserted inside the bolt to control the depth of any instrument that is inserted into the bolt. The bushing can be locked at a desired depth in the bolt through the use of an adjustable element such as a thumb screw or the like. The bushing may include gradations on its outer surface that provide a visual indication of the depth at which an instrument inserted in the bushing is to be set at. Furthermore, by one embodiment, the bushing includes a connector for connecting the bushing to an instrument and/or the bolt, and to allow precise placement of both, the bushing and the instrument within the bolt of the apparatus. For instance, the depth of the instrument can be modulated by sliding the bushing inside the bolt. This feature provides the present disclosure the advantageous ability of not requiring the entire apparatus to be repositioned, or of unlocking and re-clamping the instrument to the bolt. Specifically, the bushing can be locked at a desired depth in the bolt through the use of an adjustable element such as a thumb screw. The connector can be a simple compression-type fitting, a collar. Furthermore, the fitting may also include an adjustable element such as a screw, to attach the bushing to a specific instrument.

According to an embodiment, the above described apparatus is formed of a rigid material that is compatible with the mammalian body. For instance, the apparatus can be made of a rigid metal such as titanium, a rigid material such as ceramic, a synthetic material such as thermoplastic and the like. Furthermore, a ratio of the diameters between the proximal and distal end of the apparatus provide a slim fit, thereby allowing multiple apparatus to be used in a single intervention session. The apparatus has a robust and stable design that provisions the apparatus to be used with a plurality of motorized surgical instruments and/or in a surgical setting that requires monitoring through imaging. Additionally, the apparatus may be used either with specific instrumentation or for general surgical, diagnostic, and/or drug-delivery procedures.

Furthermore, the above described apparatus may be reusable following appropriate sterilization. According to one embodiment, the apparatus is disposed after five uses. The present disclosure provides for a method of using the above stated apparatus for the introduction of energy to a region of a brain. For instance, thermal energy may be supplied to a region of the brain using the above apparatus and the process of energy application can be monitored by magnetic resonance.

According to one embodiment, the apparatus of the present disclosure can be used to apply thermal energy to tissues in the central nervous system (e.g. the brain) of a subject. The method includes the steps of identifying a target tissue that is to be treated, inserting the apparatus into the subject in a manner that allows thermal energy to be locally delivered to the target tissue, applying thermal energy to the target tissue by transferring energy through the bolt of the apparatus. The energy exits the distal end of the bolt and heats the target tissue. Additionally, the method also includes monitoring the applied energy to ensure that the entire targeted area has been treated and that the surrounding non-targeted tissue is not damaged by thermal energy.

Furthermore, the above steps can be repeated to apply thermal energy to the target tissue that is not fully treated during the first application of thermal energy. The additional application of thermal energy may take place within a region that is accessed by a single apparatus, or by accessing the region through the use of two or more apparatus in a single subject. The use of multiple apparatus provisions for the seamless access of different regions within the target tissue. It must be appreciated that the different regions within the target tissue may be contiguous or non-contiguous.

The application of thermal energy to the target tissue region may be carried out by inserting an elongated transmitting medium into the apparatus that is secured to the subject, for example, inserted into the skull of the subject. The elongated transmitting medium is inserted through the bolt of the apparatus until a distal end of the elongated transmitting medium is operationally proximate to the target tissue. According to an embodiment, the elongated transmitting medium may be inserted by determining a safest straight path between the skull and the target tissue. The elongated transmitting medium may thereafter be inserted (through a hole drilled in the skull of the subject) towards the target tissue, until the distal end of the elongated transmitting medium is operationally proximate said target tissue.

Alternatively, the step of inserting the elongated transmitting medium may also include the step of inserting a cannula (a tube that is inserted into the subject for delivery/removal of fluids) into the hole of the apparatus, until a distal end of the cannula is operably proximate to the target. Further, the cannula is secured relative to the apparatus and the elongated transmitting medium is inserted through the cannula towards the target tissue, until the distal end of the elongated transmitting medium is operationally proximate to the target tissue.

According to an embodiment, the target tissue may be directly accessed by the insertion of an instrument including an elongated transmitting medium into the tissue. Alternatively, the target tissue may be accessed through the insertion of the elongated transmitting medium into an artery, wherein the elongated transmitting medium is fed through the artery until a distal end of the elongated transmitting medium is operationally proximate to the target tissue. Additionally, the elongated transmitting medium may be introduced to a fatty tissue region within the subject for thermal treatment.

According to an embodiment, the application of energy to the target tissue through the elongated transmitting medium may be accomplished by transmitting light, laser, collimated light, or non-collimated light, through an optical fiber. Specifically, the application of light energy may be accomplished by causing the energy to exit the distal end of the elongated medium at an angle that is greater than zero, to a longitudinal axis of the elongate transmitting medium. Furthermore, the elongated transmitting medium may be rotated around the longitudinal axis to create a shaped area of the treated tissue. The above steps of energy application and rotation of the elongated transmitting medium may be repeated until the desired target tissue region has been heated.

According to one embodiment, the application of energy may be accomplished by causing the thermal energy to exit the distal end of the transmitting medium at an angle that is approximately perpendicularly to the longitudinal axis of the elongated transmitting medium. In doing so, a disc-shaped area of treated tissue can be obtained. The thermal energy may also exit the distal end at an angle, other than being perpendicular to the longitudinal axis of the elongate transmitting medium, thereby resulting in a cone-shaped area of treated tissue. Alternatively, the thermal energy may exit the distal end along a longitudinal axis of the elongate transmitting medium.

According to an embodiment, the method of applying thermal energy by using the apparatus of the present disclosure may also include a step of monitoring the thermal energy application, in order to ensure that surrounding non-targeted tissue(s) are not damaged by heat. The monitoring can be accomplished by obtaining the temperature of the non-targeted tissue that is disposed in the vicinity of the targeted tissue. Additionally, the monitoring may also be accomplished by cycling a cooling fluid to and from the distal end of the elongated transmitting medium thereby preventing any potential damage to the surrounding non-targeted tissue.

Alternatively, the apparatus of the present disclosure can be used in combination with a magnetic field to allow monitoring of the therapeutic intervention. Accordingly, the apparatus and the corresponding method can be used for direct delivery (across the blood brain barrier) of agents, such as therapeutic or diagnostic agents, to the neural structures of the CNS of the subject.

According to one embodiment, the apparatus of the present disclosure can be used in conjunction with a magnetic resonance imaging instrument. The therapeutic intervention can be monitored either in a continuous fashion during the application of thermal energy or may be alternatively monitored in an intermittent manner throughout the application of energy to the subject.

Furthermore, by an embodiment, the apparatus can be used in conjunction with a movable static field magnet that is adapted to apply a static magnetic field in a magnetic resonance volume at a predetermined disposition relative to the static field magnet. The apparatus also includes an energy applicator that is adapted to apply energy within an energy application zone at a predetermined disposition relative to the applicator. The static field magnet can be a single-sided static field magnet that is arranged in a fashion such that a magnetic resonance volume is disposed outside of the static field magnet and is spaced from the static field magnet in a forward direction. According to an embodiment, the static field magnet is substantially smaller than the static field magnets that are utilized in magnetic resonance imaging instruments. For instance, the static field magnet may have dimensions of a meter or less and may be light enough to be moved readily by a positioning device that is of reasonable cost and proportion. Accordingly, the apparatus of the present disclosure is compact and inexpensive, such that it can be used in a clinical setting such as a physician's office or medical center.

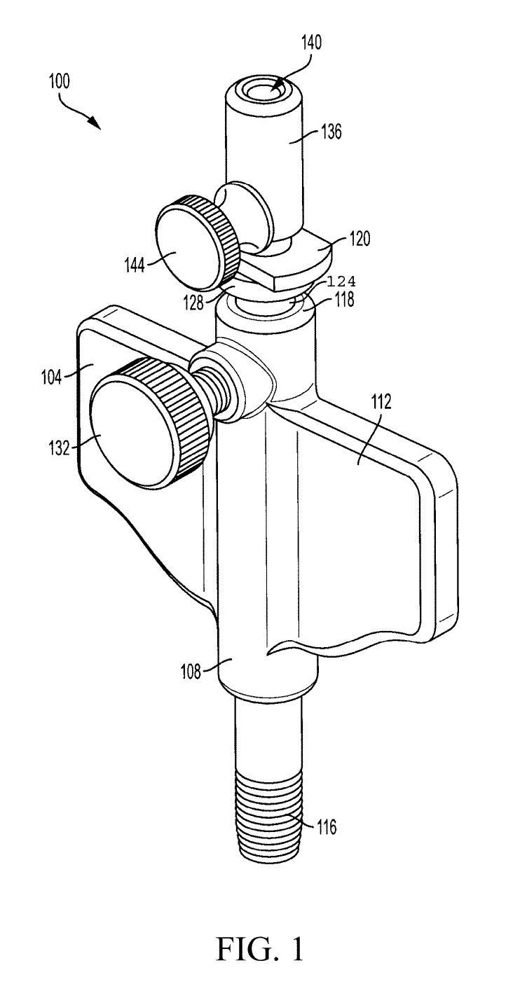

Turning now to FIG. 1, is illustrated according an embodiment, an exemplary skull mounted bolt 100. The bolt 100 is a rigid skull fixation device that is designed to provide a stable platform for inserting neurosurgical devices or instruments within a subject.

The bolt 100 includes a body 104 that is formed of a cylindrical base 108 and planar members (referred to herein as wings) 112 that extend radially outward from the cylindrical base 108. The exemplary embodiment depicted in FIG. 1 includes two wings 112 that are positioned on opposite sides of the cylindrical base 108. A first end of the cylindrical base 108 includes a tapered threaded portion 116. The cylindrical base 108 also includes an opening 118 that extends from the first end of the cylindrical base 108 to a second end of the cylindrical base 108, which is opposite to the first end.

The bolt 100 also includes a bushing 120 that is adjustable with respect to the body 104. The bushing 120 includes a cylindrical body 124 that is designed to fit within the opening 118 of the body 104. Specifically, the cylindrical body 124 has a diameter that is smaller than a diameter of the opening 118 of the cylindrical base 108, thereby allowing the cylindrical body 124 of the bushing 120 to slide within the cylindrical base 108 of the bolt 100. When the bolt 100 is assembled, the cylindrical body 124 and the opening 118 are coaxial.

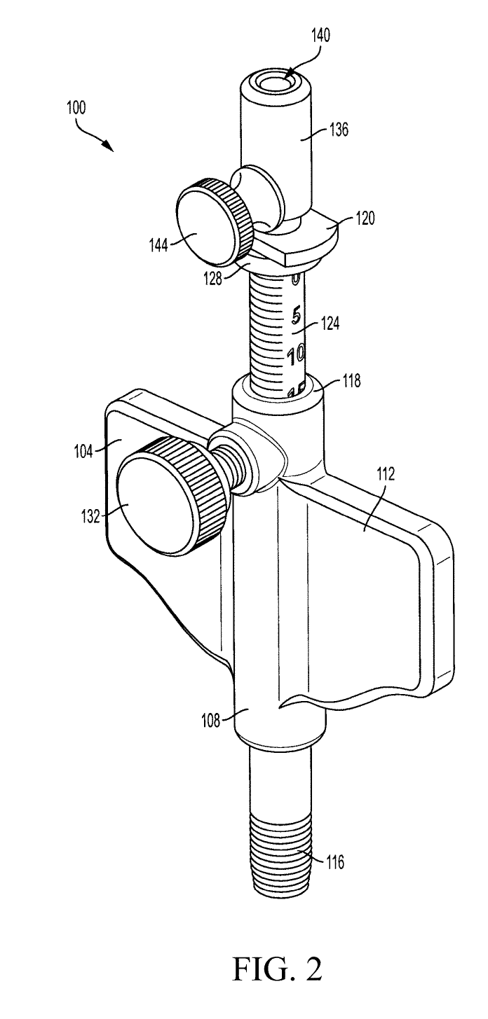

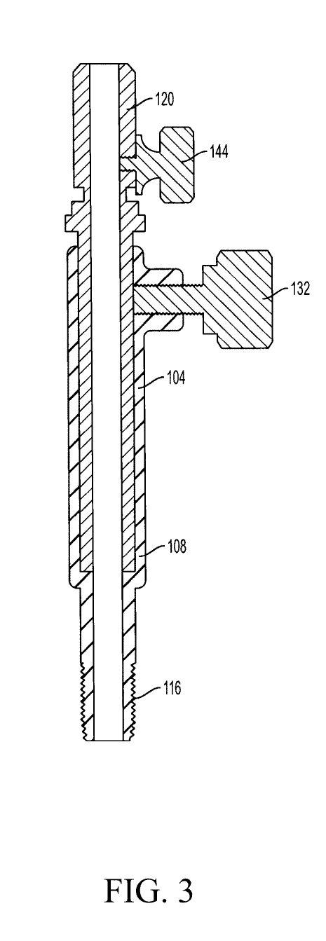

The bolt 100 further includes a fixing device (e.g., a bushing lock screw) 132, a probe locking screw 144, a stopper member 128, and a connector 136 having an opening 140. These parts of the skull mounted bolt 100 are explained in detail below with reference to FIG. 2 and FIG. 3, wherein FIG. 2 depicts an alternate view of the exemplary skull mounted bolt 100 of FIG. 1, and FIG. 3 depicts a cross-sectional view of the exemplary skull mounted bolt 100 of FIG. 1.

As depicted in FIG. 2, the cylindrical body 124 includes marked gradations. In the exemplary embodiment depicted in FIG. 2, the markings indicate a scale in millimeters. For example, the cylindrical body 124 includes a marking every millimeter, with a numeral etched on the cylindrical body 124 every fifth millimeter. However, the cylindrical body 124 may include markings for other units of measurement, such as centimeters, inches, and the like.

The cylindrical body 124 of the bushing 120 fits within the opening 118 of the cylindrical base 108, so as to assemble the bushing 120 with respect to the body 104. The bushing 120 includes a stopper 128 that is positioned above the cylindrical body 124. The stopper 128 has a diameter that is greater than the diameter of the opening 118. Thus, the cylindrical body 124 of the bushing 120 can be inserted into the opening 118 until the stopper 128 is in contact with the opening 118.

The body 104 of the bolt 100 also includes a fixing device 132 that is configured to lock the bushing 120 to the body 104. The fixing device 132 can be a thumb screw, a bushing lock screw or the like. As shown in FIG. 3, the thumb screw 132 is threaded to the cylindrical base 108, such that turning the thumb screw 132 causes it to be screwed further into the cylindrical base 108 until an end of the screw contacts the cylindrical body 124 of the bushing 120. The contact locks the bushing 120 in place with respect to the body 104 of the bolt 100. The thumb screw 132 can be turned in an opposite direction such that the end of the thumb screw 132 is backed away from, and out of contact with the cylindrical body 124, in order to reposition the bushing 120 with respect to the body 104.

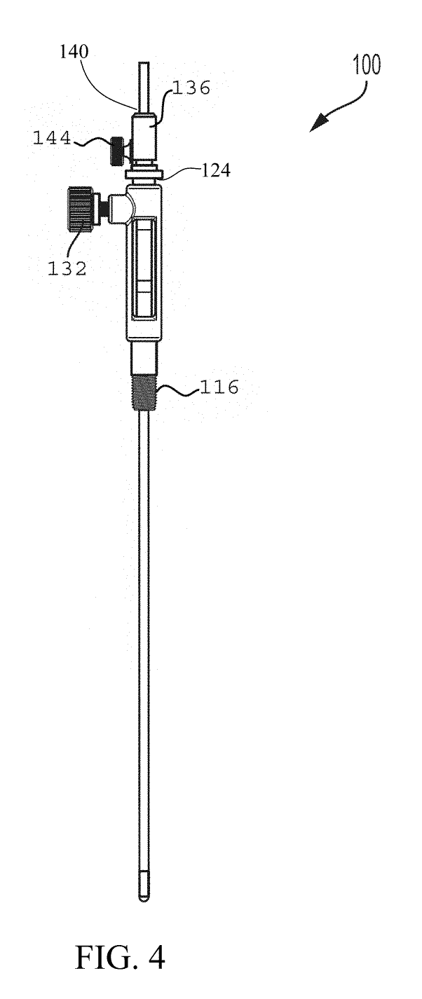

Referring to FIG. 2, the bushing 120 also includes a connector 136 that is disposed above the bushing 120. The connector 136 includes an opening 140 that extends through the bushing 120. Thus, when the bushing 120 is assembled to the body 104 of the bolt 100, the opening 140 is coaxial with the opening 118, such that a probe or other surgical instrument can be passed through the bolt 100 into the subject. Furthermore, the connector 136 includes a probe lock screw 144. The connector 136 allows a probe to be connected to the bolt 100. The functioning of the probe lock screw 144 and the thumb screw 132 with regards to the cylindrical body 124 of the bushing 120 is explained next with reference to FIG. 4.

FIG. 4 depicts according to an embodiment, a surgical probe 400 that is inserted into the skull mounted bolt 100. The surgical probe 400 is inserted through the opening 140 of the connector 136 and passes through the bushing's cylindrical body 124 and exits from the threaded portion 116 of the bolt 100. Specifically, as depicted in FIG. 4, when the probe 400 is inserted, for instance, into the skull of the subject, via the opening 140, the probe 400 is held intact with the probe lock screw 144, which operates in a manner similar to the thumb screw fixing device 132 described above. Alternatively, the probe 400 can be positioned on the connector 136 and fed through the opening 140, and then held in place by friction or a compression fitting. Therefore, by using the thumb screw 132, the bushing can be locked in place with respect to the main body 104 of the skull mounted bolt. Thereafter, adjustments in the depth of the probe 400 can be adjusted by manipulating the probe lock screw 144 without interfering or changing the state of the thumb screw 132. Alternatively, by one embodiment, the probe 400 can be positioned on the connector 136 and fed through the opening 140, and then held in place by friction or a compression fitting.

In an exemplary embodiment, the body 104 and the bushing 120 of the bolt 100 as depicted in FIG. 2 are made of titanium. The body 104 and the bushing 120 can each be produced from a titanium body by machining. The fixing device 132 and the probe lock screw 144 are made of brass, as the brass does not mark indentations on titanium. Alternatively, the bolt 100 could be made of a ceramic and/or a thermoplastic material. Furthermore, the bolt 100 can be made from materials that can be sterilized in order to allow the re-use of the bolt. Specifically, the bolt is constructed to be disposable and useful for at least a single therapeutic application.

The tapered threaded portion 116 of the bolt 100 can include a thread that self-taps into a pre-drilled hole in the skull. For instance, the threaded portion 116 can be sized to self-tap into one of a 6 mm hole, 4.5 mm hole, 3.2 mm hole and the like. However, it must be appreciated that the threaded portion 116 can be constructed so as to self-tap into holes of different sizes.

According to an embodiment, the cylindrical body 124 of the bushing 120 can be sized such that cylindrical body includes gradations for 30 mm of travel. Accordingly, the cylindrical body 124 includes an additional portion that extends beyond the 30 mm gradation, thereby enabling the fixing device 132 to be in contact at this additional portion to lock the bushing 120. Therefore, the cylindrical body 124 may have a length of 40 mm. The gradations on the cylindrical body 124 of the bushing 120 are laser etched into the cylindrical body 124.

Further, by an embodiment, the total height measured from the bottom of the body 104 to the top of the bushing 120 is 89.2 mm. The height of the threaded portion 116 is 9.8 mm. The height of the body 104, measured from the bottom of the threaded portion 116 to the top of the opening 118 is 65.0 mm. Additionally, the width of the body 104, measured from an outer edge of one of the wings 112 to the outer edge of the other wing 112, is 50.0 mm. However, it must be appreciated that each of the above exemplary dimensions can be varied as long as the resultant bolt is not inconsistent with the description herein.

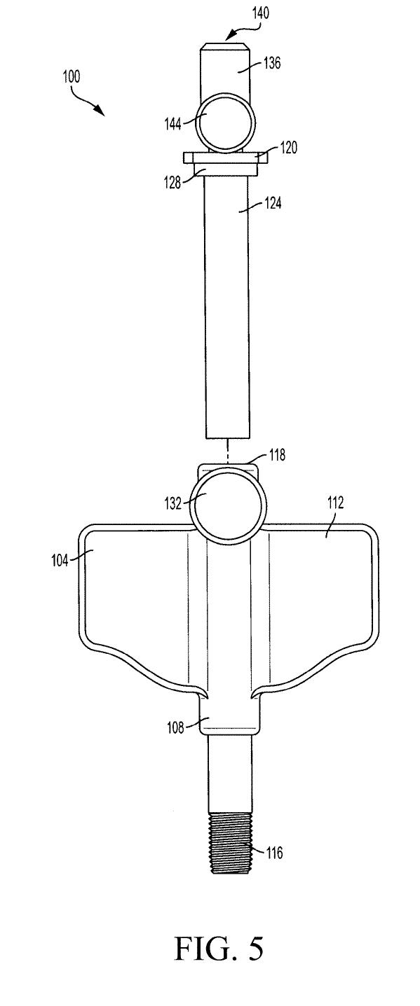

In what follows, an exemplary process of using the bolt 100 in a surgical procedure is described with reference to FIG. 5. FIG. 5 illustrates an exploded view of the bolt 100, with the body 104 being separated from the bushing 120.

Initially, the threaded portion 116 of the bolt 100 is aligned with a pre-drilled hole in the skull. As the threaded portion 116 of the bolt 100 is self-tapping, the bolt can be affixed to the skull, for instance, by utilizing the wings 112. Specifically, the wings 112 can be turned in a first direction (clockwise, for example) to thread the bolt 100 into the pre-drilled hole.

Upon threading the bolt 100 to the skull, a probe is attached to the bolt 100. According to an embodiment, the probe can be attached to the bolt by inserting a portion of the probe into the opening 140 and then feeding the probe through the opening 118 such that is passes into the skull. When the probe is at a desired position within the skull, the probe lock screw 144 can be rotated until an end of the probe lock screw 144 contacts the probe, thereby locking the probe with respect to the bushing 120 and thus the bolt 100.

Upon locking the probe to the bushing 120, the depth of the probe within the skull can be set and adjusted without unlocking the probe from the bushing 120. Specifically, the cylindrical body 124 is fully inserted into the opening 118, such that the stopper 128 contacts the opening 118. Thus, the top of the opening 118 is in level with the marking on the cylindrical body 124 indicating zero millimeters. Further, the fixing device 132 is rotated such that it does not contact the cylindrical body 124, thereby rendering the cylindrical body 124 free to move within the opening 118.

The position of the cylindrical body 124 with respect to the opening 118 can be adjusted, such that the desired marking is in level with the top of the opening 118. Once the cylindrical body 124 is in the desired position, the fixing device 132 can be rotated to lock the cylindrical body 124 in place. Note however, if further adjustments in the position of the cylindrical body 124 are required, the fixing device 132 can be loosened to enable moving the bushing 120 with respect to the body 104.

Upon completion of the surgical procedure, the probe lock screw 144 is loosened such that the probe can be removed from the bolt 100. Further, the bolt 100 is removed from the skull by gripping the wings 112 and turning the bolt 100 in a second direction (counter clockwise, for example) opposite to the first direction, until the bolt 100 disengages from the skull. Alternatively, the probe can remain attached to the bolt 100 and removed from the skull together with the bolt 100.

Alternatively, according to an embodiment, the position of the bushing 120 can be set at a desired position (for example, 5 mm) with respect to the body 104, before the probe is attached to the bushing 120. Further adjustments can be made, if necessary, after the probe is attached to the bushing 120. Accordingly, the bolt 100 described above provides the advantageous ability to easily and accurately adjust the depth of the probe by sliding the busing 120 inside the opening 118 of the body 104 of the bolt 100. Additionally, it must be appreciated that the probe does not have to be unlocked from the bolt 100 and then re-clamped when the depth of the probe is to be adjusted.

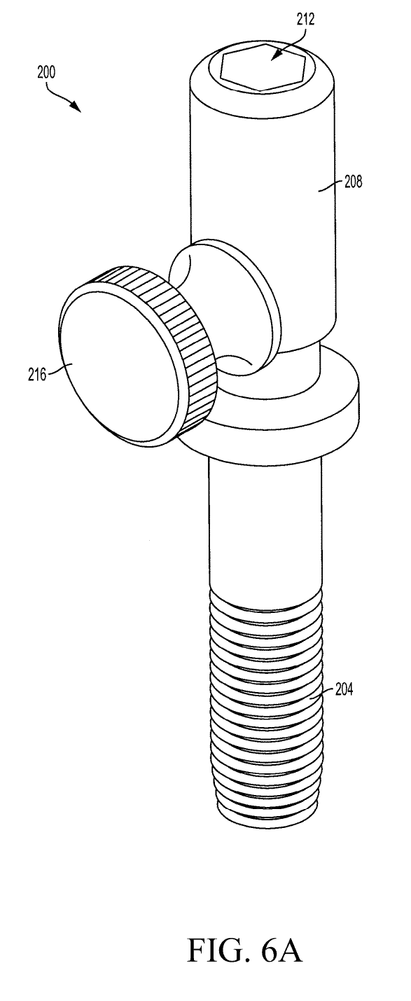

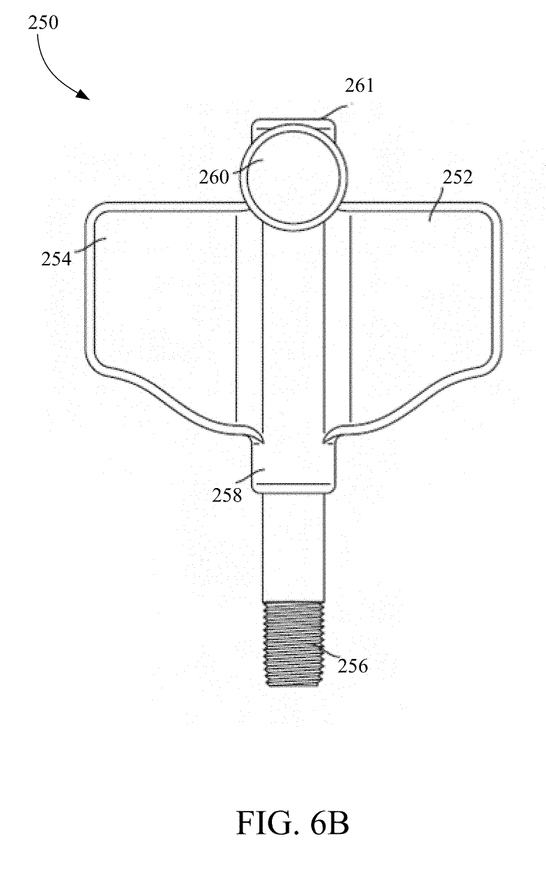

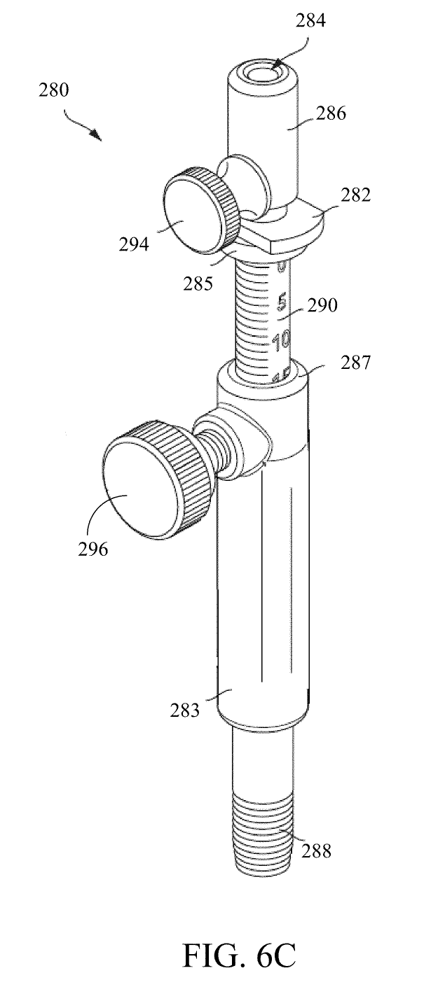

FIG. 6A depicts according to an embodiment, a skull mounted bolt 200. The bolt 200 is a rigid skull fixation device that is designed to provide a stable platform for delivering neurosurgical devices or instruments into a subject.

As shown in FIG. 6A, the bolt 200 is a one-piece device that is directly threaded into a pre-drilled hole in the skull. The bolt 200 includes a tapered threaded portion 204 on one end (referred to herein as a distal end of the bolt 200) that self-taps into the pre-drilled hole. A proximal end (the end opposite to the distal end) of the bolt 200 is sized to accept an instrument for neurological intervention that can access the target tissue through the distal end of the bolt 200. The proximal end of the bolt is dimensioned in a manner such that multiple bolts can be placed in close proximity of one another. The distal end of the bolt is designed with an internal opening such that the distal end can receive instruments having dimension of 3.3 mm or less in diameter. Furthermore, the outside dimension of the bolt 200 is not greater than 6 mm, thereby reducing potential injuries to the skull.

The bolt 200 is designed with robustness in order to support motorized surgical instruments. Specifically, the bolt 200 has a Young's modulus that is higher than 2 GPa. According to an embodiment, the threaded portion 204 of the bolt 200 can be sized to self-tap into one of a 6 mm hole, 4.5 mm hole, 3.2 mm hole and the like.

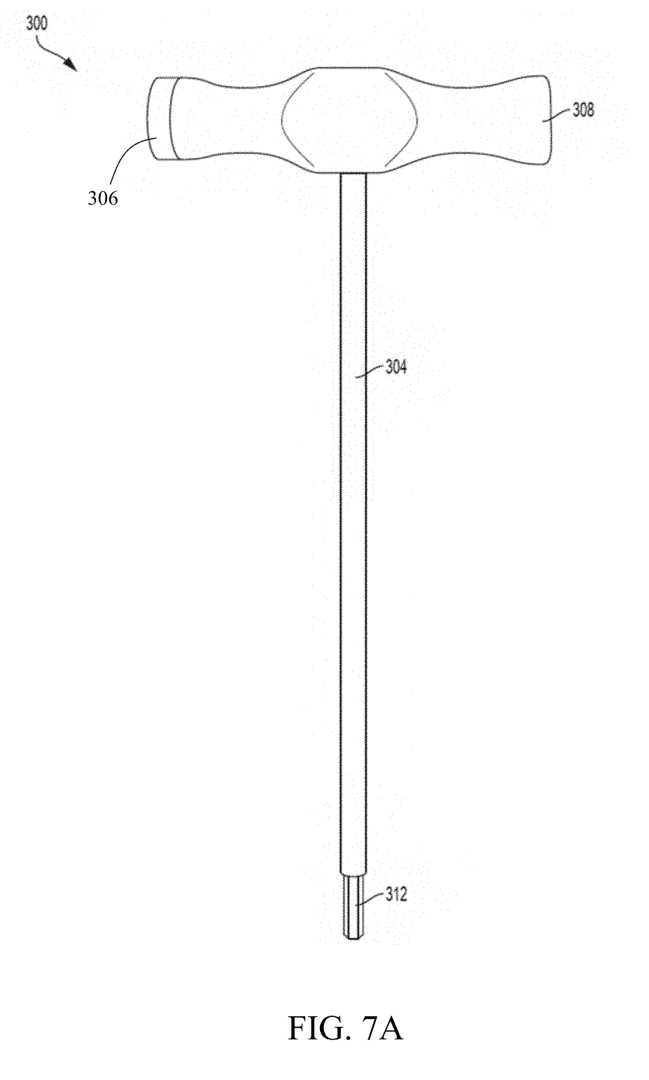

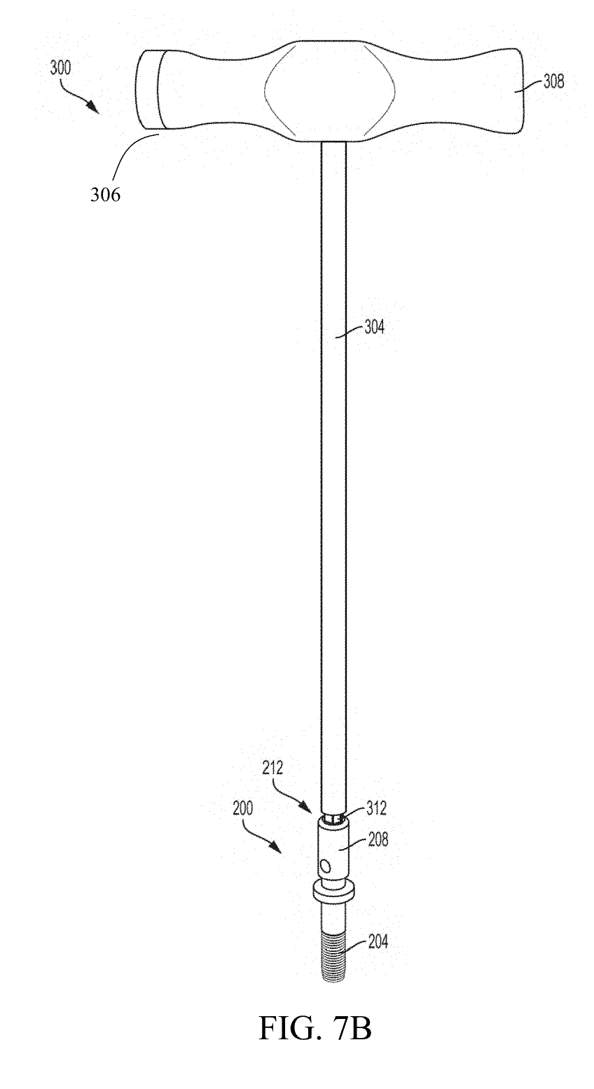

According to an embodiment, the bolt 200 includes a connector 208 and a probe lock screw 216. As shown in FIG. 6A, the bolt 200 includes an opening 212 that extends continuously from the top of the connector 208 to the end of the threaded portion 204. The width of connector 208 is designed to allow multiple devices (bolts) be placed with close proximity of one another. In an exemplary embodiment, the connector 208 can be sized to have a diameter of 6 mm, 8 mm, and the like. Further, as shown in FIG. 6A, the bolt 200 has a hexagonal shaped opening, thereby provisioning a bolt driver including a hexagonal shaped ridged portion (described later with reference to FIG. 7A) to interface/engage with the bolt 200, and further apply the required torque in order to insert the bolt 200 in the skull of a subject. Moreover, it must be appreciated that although the bolt 200 as shown in FIG. 6A has a hexagonal opening, bolts having other geometrical shapes of the opening 212 are well within the scope of the present disclosure.

A probe can be connected to the bolt 200 by inserting the probe through the opening 212 and then held in place with the probe lock screw 216. The probe lock screw 216 operates in a manner similar to the probe lock screw 144 as describe previously with reference to FIG. 2. Alternatively, the probe can be inserted through the opening 212 and held in place by friction or by a compression fitting.

In an exemplary embodiment, the bolt 200 can be made of titanium, ceramic, and/or a thermoplastic material. For instance, the bolt 200 can be machined from a titanium block. The probe lock screw 216 can be made of brass. Alternatively, the probe lock screw 216 can be made of titanium, ceramic and/or thermoplastics. According to an embodiment, a total height (measured from the bottom of the threaded portion 204 to the top of the opening 212) of the bolt 200 is 42 mm, wherein the height of the threaded portion 204 is 12 mm. It must be appreciated that each of the above described exemplary dimensions can be varied as long as the resultant bolt is not inconsistent with the description herein.

FIG. 6B depicts according to an embodiment, an exemplary skull mounted bolt 250. The bolt 250 is a rigid skull fixation device that is designed to provide a stable platform for delivering neurosurgical devices or instruments.

The bolt 250 is a one-piece device that includes a body 254 formed of a cylindrical base 258. The bolt 250 also includes planar members or wings 252 extending radially outward from the cylindrical base 258. The exemplary embodiment shown in FIG. 6B includes two wings 252 positioned on opposite sides of the cylindrical base 258. A first end of the cylindrical base 258 includes a tapered threaded portion 256. The cylindrical base 258 also includes an opening 261 that extends from a first end (top end of the bolt 250) to a second end of the cylindrical base 258 (bottom of the tapered threaded portion 256), which is opposite to the first end.

The cylindrical base 258 includes a probe lock screw 260 which is configured to hold a probe in an intact position. Specifically, when the probe is inserted into the skull via the opening 261 by a desired distance, the probe is held in place with the probe lock screw 260, which operates in a manner similar to the thumb screw fixing device 132 as described previously. In an alternative embodiment, the probe can be positioned and fed through the opening 261, and then held in place by friction or a compression fitting.

FIG. 6C depicts according to an embodiment, an exemplary skull mounted bolt 280. Note that the skull mounted bolt depicted in FIG. 6C is similar to the skull mounted bolt 100 depicted in FIG. 2. Hence in order to avoid repetition, a description of the corresponding similar parts (282, 283, 284, 285, 286, 287, 288, 290, 294, and 296) of the skull mounted bolt 280 is omitted herein. It must be appreciated that the skull mounted bolt 280 differs from the bolt 100 of FIG. 2, in that the bolt 280 does not include wings disposed on the main cylindrical body. Accordingly, the bolt 280 is more compact (and light) that the bolt 100 and provisions for multiple bolts to be placed at close proximity of one another during a surgical intervention.

According to an embodiment, the above described bolts may be affixed to the skull of the subject by using a probe driver as shown in FIG. 7A and FIG. 7B.

FIG. 7A depicts an exemplary probe driver 300 that can be used to attach the above described bolts to the skull, whereas FIG. 7B illustrates an exemplary embodiment depicting the driver of FIG. 7A being inserted into the skull mounted bolt of FIG. 6A. Referring to FIG. 7A, the driver 300 includes a shaft 304 and T-shaped handle 308. The handle 308 of the driver includes a push button 306, which is disposed at one end of the handle 308. The push-button (also referred to herein as an insert-release button) enables changing of the shaft 304 that is to be attached to the handle 308. Specifically, upon pressing the button 306, the shaft 304 is released from the handle 308, whereas upon depressing the button 306, a desired shaft may be affixed to the handle 308.

The probe driver 300 includes a ridged portion 312 disposed at a distal end of the shaft 304. The distal end of the shaft is the end that is away from the handle 308. The ridged portion 312 of the shaft enables the affixing/removing of the bolt from the skull of the subject. Specifically, the driver 300 can be inserted into the skull mounted bolt (as shown in FIG. 7B) having an opening that is of a similar shape as that of the ridged portion 314 of the driver. In doing so, the driver 300 can engage with the bolt and impart a torque to drive the bolt into the skull of the subject.

An exemplary process of using the bolt 200 in a surgical procedure includes inserting the shaft 304 of the driver 300 into the opening 212 in the top of the bolt 200 (as shown in FIG. 7B). The threaded portion 204 of the bolt 200 is aligned with a pre-drilled hole in the skull. As the threaded portion 204 is self-tapping, the handle 308 of the driver 300 can be used to turn the bolt 200 in a first direction (for example, clockwise) to thread the bolt 200 into the pre-drilled hole. Upon the bolt 200 being threaded into the skull by a sufficient distance, the driver 300 may be removed from the opening 212 in the bolt 200.

After the driver 300 is removed, a probe is attached to the bolt 200. According to an embodiment, the probe can be attached to the bolt by inserting the probe into the opening 212, and further feeding the probe through the bolt 200 into the skull. When the probe reaches the desired position, the probe lock screw 216 is turned until an end of the probe lock screw 216 contacts the probe, thereby locking the probe with respect to the bolt 200.

Additionally, after the surgical process is complete, the probe lock screw 216 can be turned such that it is no longer in contact with the probe, thereby enabling the removal of the probe from the bolt 200. The bolt 200 is then removed from the skull by re-inserting the shaft 304 of the driver 300 into the opening 212 in the top of the bolt 200, and further turning the handle 308 to thereby turn the bolt 200 in a second direction (counter clockwise, for example) opposite to the first direction.

According to an embodiment, the bolt 200 has a slim profile, thereby allowing multiple bolts to be inserted into the skull at multiple trajectories that are within close proximity of one another. The skull mounted bolt is robust, accurate, and provides a seamless way to provide stereotactic guidance, placement and fixation for the operation of instruments or devices. Additionally, the bolt is MRI compatible and has a slim fit to allow multiple trajectories. Furthermore, a slim profile of the bolt is required in order to ensure that the dimension of the bolt opening is substantially similar to the dimension of the bolt driver. Thus, the bolt and the bolt driver form an assembly that has a continuous diameter, thereby allowing the assembly to pass through a stereotactic instrument that is used to align the trajectory of the bolt. It must be appreciated that the above described bolts have a configuration that provision for seamless integration of surgical instruments, which can be used for general surgical purposes or for a specific purpose.

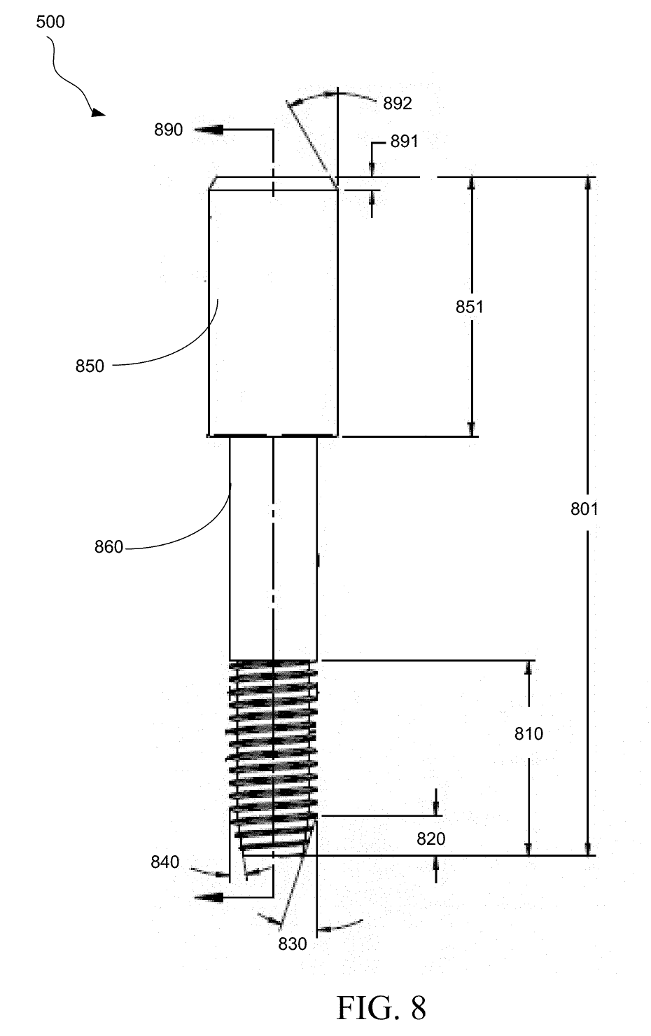

Turning now to FIG. 8 is illustrated according to one embodiment, an exemplary skull mounted bolt 500. The skull mounted bolt 500 is a rigid skull fixation device that is designed to provide a stable platform for delivering neurological devices or instruments into a subject.

As shown in FIG. 8, the bolt 500 is a one-piece device that is directly threaded into a pre-drilled hole in the skull. The bolt 500 includes a connector 850 (also referred to herein as a collar) and a cylindrical base 860. The bolt 500 includes a threaded portion of length 810 on one end (referred to herein as a distal end of the bolt 500) that self taps into the pre-dilled hole. A proximal end (the end opposite to the distal end) of the bolt 500 is sized to accept an instrument for neurological intervention that can access the target tissue through the distal end of the bolt 500. The proximal end of the bolt is dimensioned in a manner such that multiple bolts can be placed in close proximity of one another. The distal end of the bolt is designed with an internal opening such that the distal end can receive instruments having dimension of 2.2 mm or less in diameter. Furthermore, by one embodiment, the outside dimension of the bolt 500 is 4.5 mm, thereby rendering a compact size to the bolt 500 and further reducing potential injuries to the skull while performing surgical intervention.

By one embodiment, in order to ensure that the threads of the bolt 500 engage in a seamless manner into the pre-drilled hole in the skull, a portion of length 820, of the threaded portion 810 is tapered. Accordingly, the skull mounted bolt 500 includes two types of threads: major threads that lie in the un-tapered portion of the threaded portion of the bolt 500, and minor threads (of diameter less than the diameter of the major threads) that lie in the tapered portion of the threaded portion.

Further, by one embodiment, the major threads form a first angle 830 (referred to herein as a major angle) of 18° with respect to an axis 890 of the skull mounted bolt 500 (or correspondingly, as shown in FIG. 8, with an axis that is parallel to the bolt's axis). Specifically, the major angle 830 is the angle formed between a line segment connecting an outer edge of the bottom-most major thread to the outer edges of the underlying minor threads, and the axis 890. In a similar manner, as shown in FIG. 8, the minor threads form a second angle 840 (referred to herein as a minor angle) of 9° with the axis 890 of the skull mounted bolt. In other words, the minor angle is the angle formed between a line segment connecting the outer edges of the minor threads and the axis 890.

In an exemplary embodiment, a top portion 891 of the collar 850 has an inclined edge with respect to the outer surface of the collar 850 (i.e., forms an angle 892 of 30°). The bolt 500 has an opening that is similar to the opening 212 of the bolt 200 depicted in FIG. 6A. The opening extends continuously from the top of the collar 850 to the end of the threaded portion 810, and provisions for the insertion of a surgical instrument such as a probe.

Further, the bolt 500 can be made of titanium, ceramic, and/or a thermoplastic material. For instance, the bolt 500 can be machined from a titanium block. According to an embodiment, a total height 801 of the bolt 500 (measured from the bottom of the threaded portion to the top of the opening of the bolt) is 41.65 mm, wherein the collar has a length 851 of 15.9 mm, the threaded portion 810 is 12 mm and the tapered threaded portion 820 has a length of 2.5 mm. Additionally, the threaded portion of the bolt 500 includes 32 threads per inch. It must be appreciated that each of the above described exemplary dimensions can be varied as long as the resultant bolt is not inconsistent with the description herein.

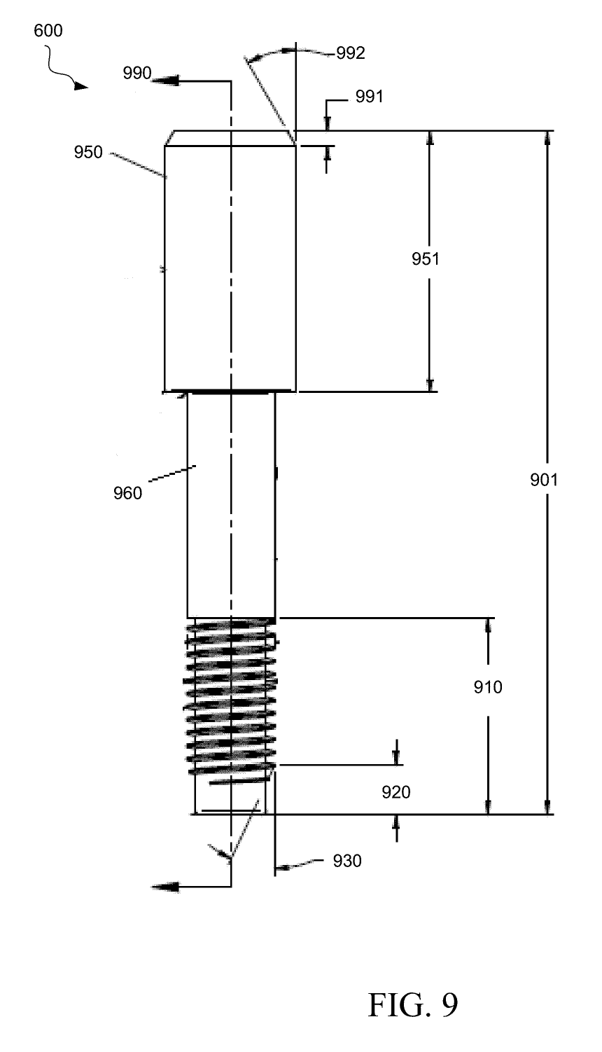

FIG. 9 illustrates according to one embodiment, an exemplary skull mounted bolt 600. The skull mounted bolt 600 is a rigid skull fixation device that is designed to provide a stable platform for delivering neurological devices or instruments into a subject.

The bolt 600 is a one-piece device that is directly threaded into a pre-drilled hole in the skull. The bolt 600 includes a connector 950 (collar) and a cylindrical base 960. The bolt 600 includes an insertion portion of length 910 that includes a threaded portion and an unthreaded portion. Specifically, by one embodiment, the insertion portion has a length 910 of 12 mm, wherein the threaded portion is 9 mm and the unthreaded portion 920 is 3 mm. By designing the bolt 600 to include the unthreaded portion 920, provides the advantageous ability of inserting the bolt into the skull of the subject in a seamless manner, when the skull wall is too thin to use a tapered minor threaded bolt (e.g., bolt 500 of FIG. 8). Furthermore, when a trajectory of inserting the bolt into the skull of the subject is inclined to skull surface, the bolt must be inserted into an angled hole, wherein the threads must “catch” (i.e. engage) to the skull surface, thereby provisioning the bolt to be threaded into the skull in a seamless manner. The unthreaded portion 920 of the bolt 600 provisions for such an insertion of the bolt tip into the hole, thereby guiding the bolt 600 before the threaded portion 910 engages with the skull surface.

The distal end of the bolt (i.e., the end where the collar is located) is designed with an internal opening such that the distal end can receive instruments having dimension of 3.3 mm or less in diameter. Furthermore, by one embodiment, the outside dimension of the bolt 600 is 4.5 mm, thereby rendering a compact size to the bolt 600 and further reducing potential injuries to the skull while performing surgical intervention. The proximal end of the bolt 600 (i.e., the end opposite to the distal end) is dimensioned in a manner such that multiple bolts can be placed in close proximity of one another.

In an exemplary embodiment, the threads of the bolt 600 form a major angle of 25° with respect to an axis 990 of the skull mounted bolt 600 (or correspondingly, as shown in FIG. 9, with an axis that is parallel to the bolt's axis). A top portion 991 of the collar 950 has an inclined edge with respect to the outer surface of the collar 950 (i.e., forms an angle 992 of) 30°). The bolt 600 has an opening, which is similar to the opening 212 of the bolt 200 depicted in FIG. 6A. The opening extends continuously from the top of the collar 950 to the end of the insertion portion 810, and provisions for the insertion of a surgical instrument such as a probe.

Further, the bolt 600 can be made of titanium, ceramic, and/or a thermoplastic material. For instance, the bolt 600 can be machined from a titanium block. According to an embodiment, a total height 901 of the bolt 600 (measured from the bottom of the insertion portion to the top of the opening of the bolt) is 41.65 mm, wherein the collar has a length 951 of 15.9 mm. Additionally, the threaded portion of the bolt 600 includes 32 threads per inch. It must be appreciated that each of the above described exemplary dimensions can be varied as long as the resultant bolt is not inconsistent with the description herein. Furthermore, the bolts 500 and 600 as illustrated in FIGS. 8 and 9 respectively, do not include a flange (i.e., a circular disk that provides support, for instance to a robotic arm, probe driver, etc.) thereby reducing the overall width of the bolt and incurring the advantageous ability of being able to slide along with a bolt driver into a stereotactic instrument that is used to align the trajectory of the bolt. Accordingly, bolts 500 and 600 can be used in a seamless manner in a surgical procedure.

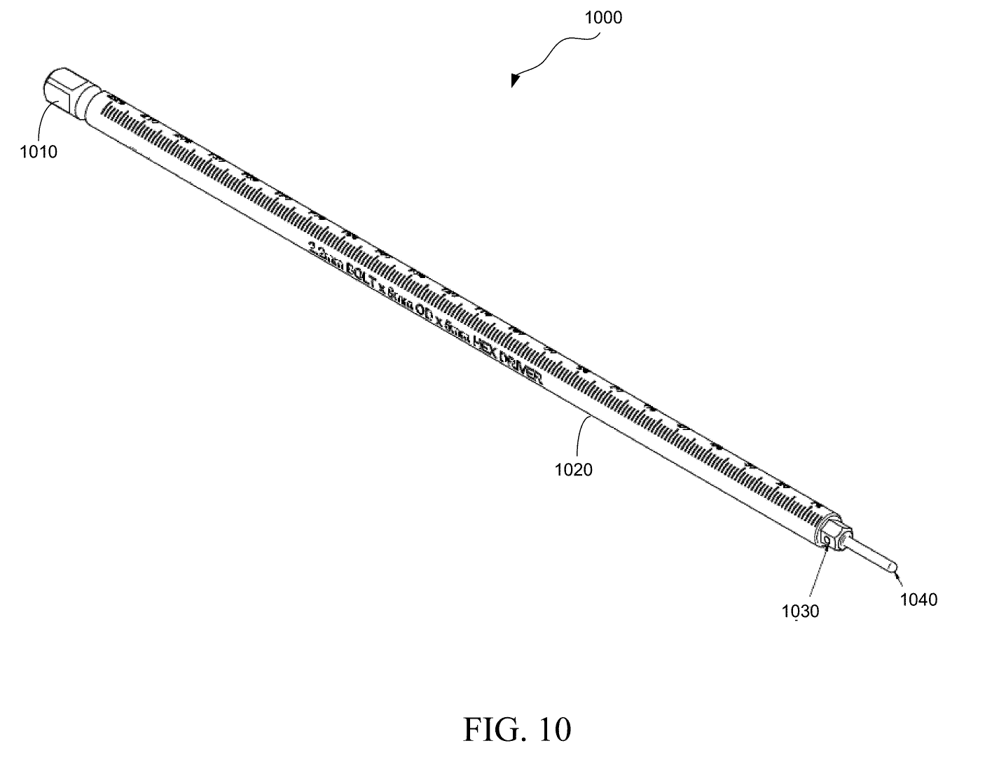

FIG. 10 depicts an exemplary probe driver 1000 that can be used to attach the above described bolts to the skull. Referring to FIG. 10, the driver 1000 includes a ridged driver head 1010 and a shaft 1020 including gradations etched on its surface. The driver head 1010 affixes into a probe driver handle that enables rotating the driver in order to affix the bolt to the subject.

The probe driver 1000 includes a plastic ridged portion 1030 that is disposed at a distal end of the shaft 1020. The distal end of the shaft is the end that is away from the head 1010. The distal end also includes a titanium shaft 1040 affixed to the ridged portion 1030. By one embodiment, the shaft 1040 is longer than a size of a socket of the bolt. Additionally, when the driver 1000 is inserted into the bolt, a plastic rod (i.e., a locking pin) may be utilized to create a small amount of interference (friction) that keeps the bolt and the driver intact while attaching the bolt to the subject.

According to one embodiment, the diameter of the opening in the cylindrical base of the above described skull mounted bolts, as well as the ratio of the first and second opening (i.e., the opening at the distal and proximal ends of the bolts) allow the apparatus to be placed in close proximity to one or more other similar apparatus. This feature provides the advantageous ability of using multiple apparatus in contiguous/non-contiguous regions in a single treatment session. Accordingly, the ability of the medical provider to apply therapeutic intervention to a larger area of the CNS (e.g., the brain) in a single session is maximized. For instance, a single therapeutic session may include 2-10 skull mounted bolts (preferably 2-5 bolts) that are disposed within close proximity of one another.

According to an embodiment, the above discussed skull mounted bolts are MRI compatible. Two or more bolts can be used in a single therapeutic session that takes place within an imaging apparatus. Accordingly, a further aspect of the present disclosure provides for methods of treating living subjects, such as human or other mammalian subjects, using a movable static field magnet adapted to apply a static field in a magnetic resonance volume.

By one embodiment, the magnet can be positioned relative to the subject so that the magnetic resonance volume, at least partially encompasses a region of the subject to be treated. A movable applicator adapted to apply energy within an energy application zone is positioned relative to the subject so that the energy application zone intersects the magnetic resonance volume within the region of the subject requiring treatment. While the static field magnet is applying the static magnetic field in the magnetic resonance volume, radio frequency signals are applied so as to elicit magnetic resonance signals from tissues of the subject in the magnetic resonance volume. The method also includes receiving these magnetic resonance signals and deriving magnetic resonance information relative to the subject's tissues in the magnetic resonance volume from the magnetic resonance signals.

According to one embodiment, the skull mounted bolts described above can be used in conjunction with a robotic probe driver. The robotic probe driver can align and position a tip of the probe at a certain distance from a target area (e.g., target tissues in the brain) that is to be treated. The probe can be used to treat various brain diseases by using thermal ablation. The diseases can range from tumors to epilepsy. According to an embodiment, the probe is aligned to the target tissue and inserted into the brain until the tip reaches the target tissue. Thereafter, laser energy is transmitted through the probe and emitted from the tip inside the target area. The energy heats the tissues causing cell death. It must be appreciated that the temperature of the probe tip can be controlled using a cooling gas and thermal monitoring.

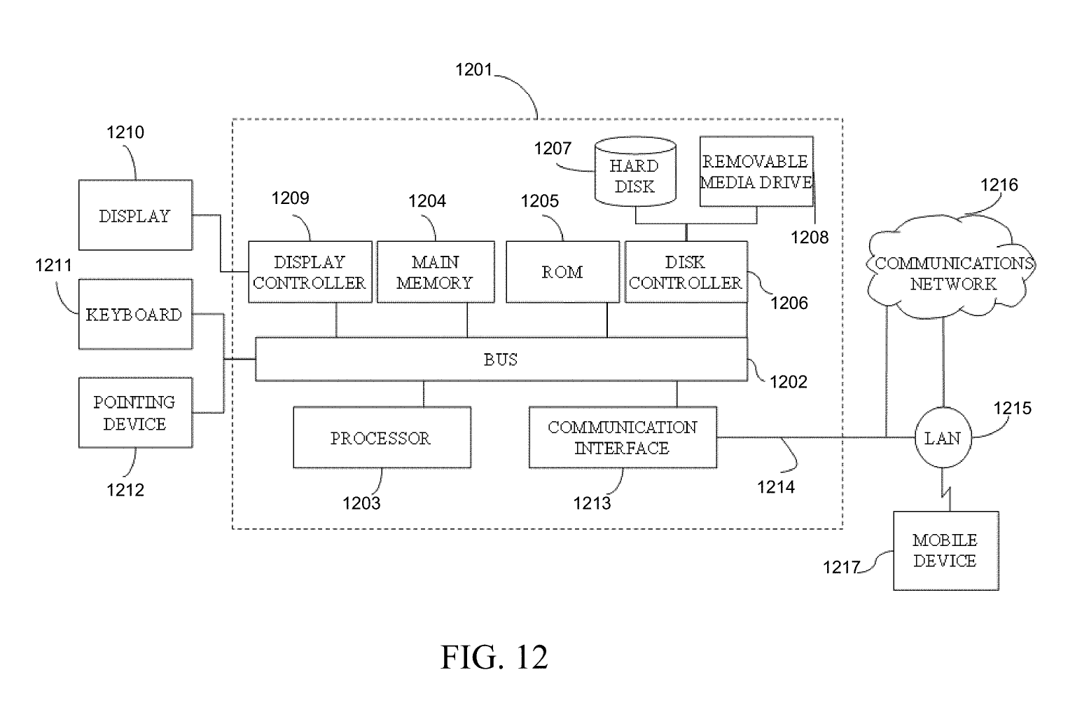

Additionally, by one embodiment, the probe driver is MRI compatible and provisions for laser emissions in multiple directions and depths without creating additional skull access. The robotic probe driver can be controlled by circuitry that includes a processor (for example processor 1203, described later with reference to FIG. 12) to control the alignment and placement of the probe within the subject. For instance, a robotic arm may be used to control the alignment and insertion of the probe as well as the skull mounted bolt.

By one embodiment, various agents may be introduced into the CNS of the subject, and in particular the brain of a subject, using the apparatus and methods of the present disclosure. A variety of agents and compositions comprising such agents can be delivered using the device, including but not limited to chemotherapeutic agents, agents for treatment of neurodegenerative disease (e.g., neurotrophic factors or neuroprotective agents), antiepileptic agents, antidepressant agents, antipsychotic agents, anti-inflammatory agents, antifibrotic agents, antianxiolytics and the like.

By one embodiment, the agents delivered using the methods and devices of the present disclosure include gene therapy by delivery of transgenes encoding certain factors into the brain, which offers great promise for treating neurodegenerative conditions such as Alzheimer's disease, Parkinson's disease and Huntington's disease. Similarly, cell-based therapies typically require quite precise placement of the cell population into the targeted region of the CNS. Delivery of these agents requires that the therapeutic composition dosage be consistently provided at precise locations in the brain to ensure that a predictable amount of the intended cell or encoded factor be delivered only to targeted regions of the brain. Such precise delivery requires delivery vectors and cells encoding transgenes to be grafted at pre-determined sites in the target brain region. The apparatus and delivery system of the present disclosure allow a precise and localized introduction of such agents into targeted regions of the brain while minimizing the invasiveness of the surgical procedure. Therefore, improvements in therapeutic efficacy can be obtained by enhancing the accurate placement of transgene-containing donor cell grafts and/or viral vectors into the brain using the apparatus and methods described herein.

Furthermore, agent delivery can be provided as a single dosage form, as a bolus or encapsulated dosage form which will release drug over time, and/or the implantation of delivery device (e.g., an osmotic pump or a catheter). Such devices for delivery of therapeutic agents that can be used in conjunction with the skull mounted bolts described herein.

As stated previously, the skull mounted bolt is robust, accurate, and provides a seamless way to provide stereotactic guidance, placement and fixation for the operation of instruments or devices. Additionally, the skull mounted bolt described herein has a slim profile, which provisions multiple bolts to be inserted into the skull within close proximity of one another.

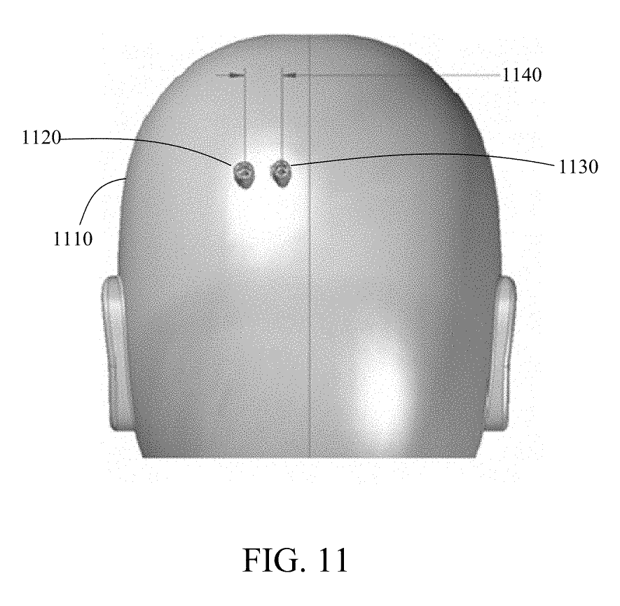

FIG. 11 illustrates an exemplary scenario depicting two skull mounted bolts 1120 and 1130, affixed to the skull 1110 of a patient. The bolts 1120 and 1130 are affixed in a manner such that the distance between the bolts (referred to herein as an inter-bolt separation distance) 1140 is at least a predetermined threshold distance. While employing multiple bolts, the inter-bolt separation is required in order to accommodate, for instance, a probe driver or a probe adapter that may be positioned over the connector portion of the bolt. Furthermore, by providing a sufficient spacing between the bolts, also provisions the surgeon with easy access to the individual bolts, as well as regions of the skull around the bolt.

By one embodiment, the inter-bolt separation distance is 16 millimeters. It must be appreciated that the magnitude of the inter-bolt separation distance is based on a type and manner in which the instrument (such as the probe adaptor) is connected to the bolt. For instance, if a probe is not fully inserted into bolt, but rather is clamped along the shaft of the probe, then the minimum inter-bolt separation distance may be lower than 16 mm.

Each of the functions of the described embodiments may be implemented by one or more processing circuits. A processing circuit includes a programmed processor (for example, processor 1203 in FIG. 12), as a processor includes circuitry. A processing circuit also includes devices such as an application-specific integrated circuit (ASIC) and circuit components that are arranged to perform the recited functions.

The various features discussed above may be implemented by a computer system (or programmable logic). FIG. 12 illustrates such a computer system 1201. In one embodiment, the computer system 1201 is a particular, special-purpose machine when the processor 1203 is programmed to perform placement of the bolt, aligning and positioning of a probe within the bolt and the like.

The computer system 1201 includes a disk controller 1206 coupled to the bus 1202 to control one or more storage devices for storing information and instructions, such as a magnetic hard disk 1207, and a removable media drive 1208 (e.g., floppy disk drive, read-only compact disc drive, read/write compact disc drive, compact disc jukebox, tape drive, and removable magneto-optical drive). The storage devices may be added to the computer system 1201 using an appropriate device interface (e.g., small computer system interface (SCSI), integrated device electronics (IDE), enhanced-IDE (E-IDE), direct memory access (DMA), or ultra-DMA).

The computer system 1201 may also include special purpose logic devices (e.g., application specific integrated circuits (ASICs)) or configurable logic devices (e.g., simple programmable logic devices (SPLDs), complex programmable logic devices (CPLDs), and field programmable gate arrays (FPGAs)).

The computer system 1201 may also include a display controller 1209 coupled to the bus 1202 to control a display 1210, for displaying information to a computer user. The computer system includes input devices, such as a keyboard 1211 and a pointing device 1212, for interacting with a computer user and providing information to the processor 1203. The pointing device 1212, for example, may be a mouse, a trackball, a finger for a touch screen sensor, or a pointing stick for communicating direction information and command selections to the processor 1203 and for controlling cursor movement on the display 1210.

The processor 1203 executes one or more sequences of one or more instructions contained in a memory, such as the main memory 1204. Such instructions may be read into the main memory 1204 from another computer readable medium, such as a hard disk 1207 or a removable media drive 1208. One or more processors in a multi-processing arrangement may also be employed to execute the sequences of instructions contained in main memory 1204. In alternative embodiments, hard-wired circuitry may be used in place of or in combination with software instructions. Thus, embodiments are not limited to any specific combination of hardware circuitry and software.

As stated above, the computer system 1201 includes at least one computer readable medium or memory for holding instructions programmed according to any of the teachings of the present disclosure and for containing data structures, tables, records, or other data described herein. Examples of computer readable media are compact discs, hard disks, floppy disks, tape, magneto-optical disks, PROMs (EPROM, EEPROM, flash EPROM), DRAM, SRAM, SDRAM, or any other magnetic medium, compact discs (e.g., CD-ROM), or any other optical medium, punch cards, paper tape, or other physical medium with patterns of holes.

Stored on any one or on a combination of computer readable media, the present disclosure includes software for controlling the computer system 1201, for driving a device or devices for implementing the features of the present disclosure, and for enabling the computer system 1201 to interact with a human user. Such software may include, but is not limited to, device drivers, operating systems, and applications software. Such computer readable media further includes the computer program product of the present disclosure for performing all or a portion (if processing is distributed) of the processing performed in implementing any portion of the present disclosure.

The computer code devices of the present embodiments may be any interpretable or executable code mechanism, including but not limited to scripts, interpretable programs, dynamic link libraries (DLLs), Java classes, and complete executable programs. Moreover, parts of the processing of the present embodiments may be distributed for better performance, reliability, and/or cost.

The term “computer readable medium” as used herein refers to any non-transitory medium that participates in providing instructions to the processor 1203 for execution. A computer readable medium may take many forms, including but not limited to, non-volatile media or volatile media. Non-volatile media includes, for example, optical, magnetic disks, and magneto-optical disks, such as the hard disk 1207 or the removable media drive 1208. Volatile media includes dynamic memory, such as the main memory 1204. Transmission media, on the contrary, includes coaxial cables, copper wire and fiber optics, including the wires that make up the bus 1202. Transmission media also may also take the form of acoustic or light waves, such as those generated during radio wave and infrared data communications.

Various forms of computer readable media may be involved in carrying out one or more sequences of one or more instructions to processor 1203 for execution. For example, the instructions may initially be carried on a magnetic disk of a remote computer. The remote computer can load the instructions for implementing all or a portion of the present disclosure remotely into a dynamic memory and send the instructions over a telephone line using a modem. A modem local to the computer system 1201 may receive the data on the telephone line and place the data on the bus 1202. The bus 1202 carries the data to the main memory 1204, from which the processor 1203 retrieves and executes the instructions. The instructions received by the main memory 1204 may optionally be stored on storage device 1207 or 1208 either before or after execution by processor 1203.

The computer system 1201 also includes a communication interface 1213 coupled to the bus 1202. The communication interface 1213 provides a two-way data communication coupling to a network link 1214 that is connected to, for example, a local area network (LAN) 1215, or to another communications network 1216 such as the Internet. For example, the communication interface 1213 may be a network interface card to attach to any packet switched LAN. As another example, the communication interface 1213 may be an integrated services digital network (ISDN) card. Wireless links may also be implemented. In any such implementation, the communication interface 1213 sends and receives electrical, electromagnetic or optical signals that carry digital data streams representing various types of information.

The network link 1214 typically provides data communication through one or more networks to other data devices. For example, the network link 1214 may provide a connection to another computer through a local network 1215 (e.g., a LAN) or through equipment operated by a service provider, which provides communication services through a communications network 1216. The local network 1214 and the communications network 1216 use, for example, electrical, electromagnetic, or optical signals that carry digital data streams, and the associated physical layer (e.g., CAT 5 cable, coaxial cable, optical fiber, etc.). The signals through the various networks and the signals on the network link 1214 and through the communication interface 1213, which carry the digital data to and from the computer system 1201 may be implemented in baseband signals, or carrier wave based signals.

The baseband signals convey the digital data as unmodulated electrical pulses that are descriptive of a stream of digital data bits, where the term “bits” is to be construed broadly to mean symbol, where each symbol conveys at least one or more information bits. The digital data may also be used to modulate a carrier wave, such as with amplitude, phase and/or frequency shift keyed signals that are propagated over a conductive media, or transmitted as electromagnetic waves through a propagation medium. Thus, the digital data may be sent as unmodulated baseband data through a “wired” communication channel and/or sent within a predetermined frequency band, different than baseband, by modulating a carrier wave. The computer system 1201 can transmit and receive data, including program code, through the network(s) 1215 and 1216, the network link 1214 and the communication interface 1213. Moreover, the network link 1214 may provide a connection through a LAN 1215 to a mobile device 1217 such as a personal digital assistant (PDA) laptop computer, or cellular telephone.

While aspects of the present disclosure have been described in conjunction with the specific embodiments thereof that are proposed as examples, alternatives, modifications, and variations to the examples may be made. It should be noted that, as used in the specification and the appended claims, the singular forms “a,” “an,” and “the” include plural referents unless the context clearly dictates otherwise.

Furthermore, the foregoing discussion discloses and describes merely exemplary embodiments of the present invention. As will be understood by those skilled in the art, the present disclosure may be embodied in other specific forms without departing from the spirit or essential characteristics thereof. Accordingly, the present disclosure is intended to be illustrative and not limiting of the scope, as well as the claims. The disclosure, including any readily discernible variants of the teachings herein, defines in part, the scope of the foregoing claim terminology such that no subject matter is dedicated to the public.

1. A device comprising:
a cylindrical body including a tapered threaded portion that screws into a pre-drilled hole in a subject; and
a connector including a probe lock-screw, disposed on the cylindrical body at an end opposite to the tapered threaded portion, wherein the connector includes an opening to receive a probe, the probe being inserted into the subject via the device, and wherein a depth of insertion of the probe within the subject is adjusted by the probe lock-screw.

2. The device of claim 1, wherein a length of the tapered threaded portion is 12 millimeters, a diameter of the opening of the connector is at most 3 millimeters, and an outer diameter of the connector is at most 6 millimeters.

3. The device of claim 1, wherein a length of the device from the opening to the bottom of the tapered threaded portion is 42 millimeters.

4. The device of claim 1, wherein the pre-drilled hole in the subject has a diameter of one of 6 millimeters, 4.5 millimeters, and 3.2 millimeters, and a driver including a shaft and a T-shaped handle screws the device into the pre-drilled hole in the subject upon turning the T-shaped handle in a first direction.

5. A device comprising:
a cylindrical base including a tapered threaded portion, a lock screw, and a first opening, the first opening being disposed at an end opposite to the tapered threaded portion;
a bushing including a cylindrical body and a stopper, the cylindrical body being designed to slide within the first opening of the cylindrical base, and wherein a depth of the bushing within the cylindrical base is adjusted by the lock screw; and
a connector including a probe lock-screw, disposed above the bushing, wherein the connector includes a second opening to receive a probe, the probe being inserted into a subject via the device, and wherein a depth of insertion of the probe within the subject is controlled by adjusting at least one of the probe lock-screw and the lock screw.

6. The device of claim 5, further comprising:
a pair of planar members disposed on the cylindrical base and designed to screw the tapered threaded portion of the device into a pre-drilled hole in a subject upon being rotated in a first direction.

7. The device of claim 5, wherein the stopper is designed to prevent the bushing from sliding completely within the cylindrical base by having an outer diameter of the stopper greater than a diameter of the first opening of the cylindrical base.

8. The device of claim 5, wherein the cylindrical body of the bushing and the cylindrical base of the device are made of titanium, and the probe lock-screw and the lock screw are made of brass.

9. The device of claim 5, wherein a height of the cylindrical base measured from the bottom of the tapered threaded portion to the first opening is 65 millimeters, and wherein the height of the tapered threaded portion is 9.8 millimeters, and wherein a width of the device measured from an outer edge of one of the planer members to the outer edge of the other planar member is 50 millimeters.

10. The device of claim 5, wherein a diameter of the second opening of the connector is at most 3 millimeters and an outer diameter of the connector is at most 6 millimeters, and a diameter of the cylindrical body of the bushing is less than a diameter of the cylindrical base of the device, the cylindrical body of the bushing including laser etched gradations that indicate a depth of insertion of the probe within the subject.

11. A method of performing neurological intervention using a device including a cylindrical body that has a tapered threaded portion, and a connector disposed on the cylindrical body of the device, the method comprising:
fastening the device into a pre-drilled hole in a subject, by inserting a shaft of a driver in an opening of the connector and rotating a handle of the driver in a first direction;
aligning and inserting a probe through the fastened device, the probe being inserted through the opening of the connector;
controlling a depth of insertion of the probe within the subject by adjusting a probe lock-screw disposed on the connector, the probe lock-screw being designed to fasten the probe with respect to the device; and
delivering an agent through the attached probe to treat target tissues within the subject.

12. The method of claim 11, further comprising:
transmitting laser energy through the inserted probe, to heat a target tissue within the subject;
controlling a temperature of the probe tip by delivering a cooling gas; and
monitoring the transmitting and the controlling by MRI; and
fastening a second device adjacent to the fastened device, the second device being substantially structurally similar to the fastened device

13. The method of claim 11, wherein the delivered agent is one of a chemotherapeutic agent, antiepileptic agent, antidepressant agent, antipsychotic agent, anti-inflammatory agent, antifibrotic agent, and an antianxiolytics agent.

14. The method of claim 11, wherein the fastening is performed by inserting the shaft of the driver in an opening of a connector disposed on the second device, and rotating the handle of the driver in the first direction.

15. The method of claim 14, further comprising:
inserting a second probe through the second fastened device, the second probe being inserted through the opening of the connector disposed on the second device.
